(19) United States
(12) Patent Application Publication
(10) Pub. No.: US 2023/0059958 A1
(43) Pub. Date: Feb. 23, 2023

(54) ANTIMICROBIAL BLADDER ADDITIVES SYSTEM AND METHOD

(71) Applicant: Donald Griffith, Houston, TX (US)

(72) Inventor: Donald Griffith, Houston, TX (US)

(21) Appl. No.: 17/410,613

(22) Filed: Aug. 24, 2021

Related U.S. Application Data
(60) Provisional application No. 63/232,288, filed on Aug. 12, 2021.

(51) Int. Cl. A61K 33/18; A01N 59/12; A01N 59/00; A61K 33/40; A61K 31/7034; A61K 31/192; A61K 47/14; A01N 59/16; A61K 33/38; A01N 43/08; A01N 37/40; A61K 31/16; A01P 1/00

(52) U.S. Cl. CPC A61K 33/18; A01N 59/12; A01N 59/00; A61K 33/40; A61K 31/7034; A61K 31/192; A61K 47/14; A01N 59/16; A61K 33/38; A01N 43/08; A01N 37/40; A61K 31/16; A01P 1/00

(57) ABSTRACT

An antimicrobial composition includes an aqueous and/or ethanolic solution of iodine and hydrogen peroxide, which is acidified. The acidifying agent may be aspirin, hydrochloric acid, or other acidic agent tolerable to the body. The composition may also include a witch hazel extract, or alternately hamamelitannin, gallic acid, or other components thereof that effect quorum signaling inhibition that blocks and erodes microbe-induced biofilm on cellular and device surfaces. Acetohydroxamic acid may be combined with the composition as a counter to microbe-induced alkalinization, particularly in urine. Moreover, a metal, halogen, and combinations of these, such as a silver colloid, may be added as a secondary microbicidal agent. The antimicrobial composition may be applied topically to or instilled through an indwelling urinary catheter or applied as a device surface-coating on other medical devices.

### CROSS-REFERENCE TO RELATED APPLICATION

The present application is a conversion of and has benefit of priority of the following application, which is co-pending and has at least one same inventor of the present application: U.S. Provisional Patent Application No. 63/232,288, titled “Antimicrobial Bladder Additives System and Method”, filed Aug. 12, 2021.

### TECHNICAL FIELD

The invention generally relates to elemental compositions formulated for cleansing and/or antimicrobial actions on biological and inanimate surfaces. More particularly, the invention relates to combinations of halogens, such as for nonexclusive example iodine, chlorine, bromine and others, and/or oxygen and oxygen compounds, and/or metals such as for nonexclusive example gold, silver, copper, zinc and others, deliverable as [1] topical cleansing and antimicrobial additives applied to multiple anatomical locations, [2] inanimate surface cleansing antimicrobial, and as [3] surface coating on both permanent and temporary devices, and/or [4] otherwise. Further enhancements by additional additives are included such as to prevent and/or erode biofilm formation and/or functionalities, and these may be dependent on the biological organ or tissue of application or other use.

### BACKGROUND

Bacterial, viral and fungi and other microbial infections are a major concern in delivery of healthcare. Such infections may present serious consequences, even life-threatening consequences in some instances, to some patients. Chronically disabled and aged patients are particularly vulnerable to such infections. Various precautions and remedies exist, most notably cleansing and antibiotics. Effectiveness of these may be limited, however, in certain conditions. New antimicrobial compounds as surface or fluid additives would be advantageous. It is noteworthy that the World Health Organization [WHO] website reports that no new antimicrobial agents have been commercialized in the past 40 years.

Many patients suffer from microbial and viral infections. Aging and disabled individuals commonly suffer from conditions (e.g., neurological, physical or cognitive) that are particularly susceptible to infections, for example, to urinary bacterial colonizations and or other complications that arise as consequence of one or more disabling conditions and the management of the disabled condition. Normal, periodic, volitional urinary bladder continence is often disrupted in the aging and disabled. Aging and childbirth can weaken the pelvic floor muscles in females causing incontinence issues. In males over the age of about 50, testosterone production slowly enlarges the prostate gland and slowly constricts and closes the urethral channel within the ‘donut-shaped’ prostate gland leading to urinary retention (i.e., inability to urinate). As can be understood, certain disabilities also may cause incontinence concerns. Many organs undergo similar aging-induced changes that make the organ susceptible to colonization by all types of microbial agents (e.g., bacteria, viruses and fungi).

Surgical procedures, such as transurethral resection of prostate gland, radical prostatectomy, pelvic floor reconstruction, use of artificial urinary sphincters, and others, may successfully rehabilitate bladder and urethral channel functions in some patients. Moreover, pads, pull-ups, and diapers (e.g., used mostly by females, but also by males), or indwelling urinary catheters (e.g., used mostly by males, whose use may involve suprapubic (abdominal) or urethral catheters), can be helpful for incontinence or urinary retention. Urinary catheter users develop catheter associated urinary tract infections (“CAUTI”) at a rate of approximately 5% of users/day. Thus, virtually all chronic urinary catheter users have chronic CAUTI. The National Institutes of Health (NIH), Center for Disease Control (CDC) and Food and Drug Administration (FDA) discourage preventive use of antibiotics in asymptomatic urinary catheter-using patients but favor use of such agents when urinary colonization is symptomatic. Antibiotics commonly lead to mutant ‘resistant’ strains of urinary pathogens. These and other options, nevertheless, can lead to symptomatic, life-altering and life-threatening infection problems.

In patients with long-term catheter use (e.g., typically 15+ days), catheter associated urinary tract infections are particularly a problem. NIH, CDC, and FDA have all identified CAUTI as one of the most common and expensive infectious diseases in the United States. Prophylactic use of antibiotics to treat these infections has been discouraged, because of evolution of antibiotic-resistant microbes. Microbial colonization risks of indwelling catheters, therefore, remain an unsolved clinical problem in a growing, aged population. The infection risks are similar regardless of whether the catheter is placed abdominally or transurethrally.

Causes of CAUTI include microbial attachment of skin microbes to external surfaces of indwelling catheters, followed by explosive growth and concurrent biofilm formation. Microbes commonly adhere to cellular and inanimate surfaces and then create and enshroud themselves within a protective biofilm. Daily migration of colony-rich biofilms, principally on catheter external surfaces toward the bladder lumen, induces colonization of bladder urine. Colonized bladder urine occurs at a rate of approximately 5% of catheter users per day. Thus, virtually 100% of chronic-use catheters have colonization after ˜20+ days of indwelling catheter use. Consequently, long-term catheter users typically have colonized urine for as long as the catheter is indwelling.

It is noteworthy that urine is the excretory pathway for muscle (i.e., proteinaceous) and bone (i.e., calcium and phosphorus) waste resulting in high urinary concentrations of urea and high concentrations of calcium and phosphorus dissolved in urine. Some bacterial species (e.g., all Proteus species and some strains of Klebsiella and Pseudomonas) make urease, an enzyme that interacts with urea to robustly and quickly alkalinize urine. Alkaline urine triggers precipitation of calcium-phosphate crystals which over a few days attach and grow onto catheter surfaces as encrustations and/or as bladder stones.

Use of systemic antibiotics and antibiotic catheter coatings has been discouraged, for example, by FDA, NIH and CDC, because multiple trials have shown little continued sterility and frequent development of mutant microbial species that are or become resistant to antibiotics. It has become apparent that many of the microbial species that cause CAUTI owe much of their pathogenicity to quorum signaling and biofilm formation. Quorum signaling is a process in which bacteria communicate with each other by secreting and sensing diffusible signaling molecules called “autoinducers.” When these signaling molecules exceed a threshold concentration level, they auto-stimulate genes that enable bacteria to behave as a multicellular population, to benefit their survival. Staphylococci bacteria, as an example, create autoinducers that upregulate the expression of multiple toxins and enhance the formation of biofilms. These phenotypes are the hallmark of staph pathogenesis and are a cause of resistant and persistent infections and even death.

Acetohydroxamic acid (AHA) is an FDA certified, water-soluble, oral, therapeutic agent that is excreted in urine where it blocks urinary microbe generated urease thereby allowing urine to maintain its physiologic acidic pH. Witch hazel is a botanical native to the Northeast USA, among other locations, which includes hamamelitannin, gallic acid, and perhaps other agents that effect quorum signaling inhibition induced by Staphylococcal and multiple other microbial species and strains. Metals, such as for example, gold, silver, copper, zinc, and others, are naturally microbiocidal as are halogens (chlorine, bromine, iodine, etc.) and oxygen. Metals and halogens may be less susceptible to microbial mutations and emergence of resistance microbial species than are the man-made synthetic antibiotics.

Consequently, a significant and serious need exists for suitable new antimicrobial compounds and uses thereof. It would, therefore, be a significant improvement in the art and technology to provide new microbiocidal compounds and uses thereof. It would further be an improvement to provide new microbiocidal compounds for bladder rinsing, as well as mucous and particulate cleansing to use with urinary catheters, as well as other devices. It would also be a further improvement to provide new or enhanced antimicrobial compounds and/or new or revised methods of use that do not significantly lead to resistant microbes and/or overuse of conventional antibiotics and similar conventional antimicrobial options.

### SUMMARY

Embodiments of the invention include elemental compositions formulated for cleansing and/or antimicrobial actions on biological and inanimate surfaces. Non-exclusive embodiments include combinations of halogens, such as iodine, chlorine, and bromine and metals such as gold, silver, copper and zinc and/or oxygen and oxygen compounds, deliverable as topical cleansing and antimicrobial additives applied to multiple anatomical surface locations, and inanimate surfaces as both cleansing and antimicrobial, surface coatings on both permanent and temporary devices and/or other uses. Non-exclusive additives in embodiments include acetohydroxamic acid (AHA) and/or witch hazel concentrate. AHA blocks microbial urease-induced alkalinization and thereby supports urinary acidification thereby reducing risk of calcium-phosphate crystallization and stone formation. AHA maintains physiological acidification of urine and thereby potentiates further added acidification that enhances the functionality of elemental agents, such as iodine and oxygen. Witch hazel concentrate (WH) can block new biofilm formation and erode existing biofilm formation induced by several types of urinary pathogens. Witch hazel concentrate is functional as a dissolved urinary additive and as allopathic surface coating. Other additives are possible, such as for non-exclusive example, metals like gold, silver, copper, zinc, and others, and/or other additives.

An embodiment of the invention is an antimicrobial bladder additive liquid composition including iodine and hydrogen peroxide.

Another embodiment of the invention is acidification of the iodine/hydrogen peroxide mixture to a pH of approximately 1.8-2.8

Another embodiment of the invention is a method of antimicrobial action that includes providing an aqueous molecular [non-ionic, non-polymerized] I² iodine, and providing an aqueous hydrogen peroxide to the aqueous iodine for a first composition, and acidifying the first composition.

Another embodiment of the invention includes addition of witch hazel concentrate or its most active botanical ingredients, consisting of hamamelitannin and/or gallic acids. These agents prevent and erode microbe induced biofilm formation on prosthetic and cellular surfaces thereby exposing adherent microbes to the killing effect of iodine and hydrogen peroxide.

Yet another embodiment of the invention includes addition of acetohydroxamic acid [AHA]. AHA serves as a bladder additive formulation to prevent microbe induced alkalinity of urine that serves to negate the acidic aqueous bladder additive.

Yet another embodiment of the invention includes additions, substitutions, or omissions to an acidic iodine and hydrogen peroxide combination particularly targeted for use in or for a particular anatomical organ or a particular tissue coating.

### DETAILED DESCRIPTION

Embodiments include iodine solution and hydrogen peroxide, as well as other formulations. As non-exclusive example, embodiments also can include acidifying agents, such as acetohydroxamic acid (AHA), and/or organic extracts, such as Witch hazel extracts (WH), comprising hamamelitannin and/or gallic acid, combined with the iodine and hydrogen peroxide. The novel combination has been determined to be effective as an antimicrobial, for topical applications, including to surfaces of urinary catheters and devices, and as a topical rinse of the bladder lumenal mucosa, skin and other organs. Through benchtop experimentation, the hydrogen peroxide+molecular iodine+AHA+WH+acidification to pH 1.5-2.5 combination has been shown to be more effective as an antibacterial than the individual components. The formulations may take a variety of forms, including liquids, rinse, sprays, ointments, creams, colloids, and others.

### Hydrogen Peroxide

Hydrogen peroxide is a compound with the formula H₂O₂. It is a liquid that is slightly more viscous than water. Hydrogen peroxide has been used as an oxidizer, bleaching agent and topical antiseptic. As a topical antiseptic, hydrogen peroxide has been used as a topical “wipe” on inanimate work surfaces and topically on skin and selected organs (i.e., eyes, ears, etc.) in humans and other animals. Hydrogen peroxides with pH of about 4.0 are generally available over the counter as 3% topical cleansing agent for human skin.

Hydrogen peroxide has not been used systemically for internal organs as antimicrobial. It has been determined that antimicrobial effectiveness of hydrogen peroxide is very dependent on pH, as is steady blood pH of about 7.0. Hydrogen peroxide has not previously been used as a bladder additive, though hydrogen peroxide has been discovered to be naturally present bladder urine having been produced by bladder mucosa; thus, H₂O₂ is present in physiological urine in small concentrations.

Dilute concentrations of hydrogen peroxide [2-3%] that are acidic are potent antimicrobial agents, such as, for example, in bladder and urine applications. A solution of 3% hydrogen peroxide is combined with an acid, for non-exclusive example, acetylsalicylic acid (ASA), i.e., aspirin, or hydrochloric acid, or acetic acid or or other acid. Desired acidification is to a pH of about 1.5 to about 2.5. The acidified solution has been found to be an effective and potent antimicrobial, which may be employed topically or introduced internally [into specific organs, spaces, or cavities] of humans and animals, such as for bladder rinse and as topical application to skin and/or catheter or other permanent or temporary device surfaces.

### Iodine

Aqueous iodine in about 3% and 6% solutions [as tincture of iodine, i.e. dissolved first in ethanol and then in water] or as Lugol solution [i.e. a mixture of I² and potassium ionized iodine] are commercially available as a topical skin cleansing agents. Both formulations are used to clean and disinfect inanimate and cellular surfaces. Only the I² moiety is antibacterial. The I² moiety can be increased by solubilizing the iodine in ethanol and/or by acidifying the aqueous mixture. The solution is often found in mouthwash or topical wound treatment for small wounds, and similar product form. The solution typically has pH of about 4.5.

Aqueous iodine has not generally been used as a topical bladder rinse or for other topical internal bodily use. It has been found that the molecular iodine component (I²) of the aqueous solution is the only antimicrobial component. The percent of molecular iodine in the aqueous solution can be increased by adding iodine to ethanol and by mixing the ethanol solubilized I² with water and by increasing acidity of the aqueous solution, for example by lowering the mixture to pH of about 1.5 to about 2.0. Aspirin, hydrochloric acid, acetic acid, benzoic or other acids may lower to this pH. At such acidic pH, the acidic aqueous solution has been found to be a tolerable antimicrobial solution for introduction into the bladder, as well as for topical application to skin, and other cellular or inanimate device surfaces.

### Combined Acidic Agents

Combination of acidic aqueous combinations of hydrogen peroxide and iodine have been found to be a very effective formulation for antimicrobial effects. Furthermore, the combination has been found to be tolerable for use on and in connection with the human body, such as for a urinary bladder rinse. Certain lab results follow:

Experiment 1 (05/14/21): Aqueous Iodine plus H₂O₂

Organism: Staphylococcus aureus

Growth media: Luria Broth

| Iodine Dilution | H2O2 Dilution | Interpretation |
|---|---|---|
| 1:10 | 1:2 | Susceptible |
| 1:10 | 1:50 | Susceptible |
| 1:10 | 1:100 | Susceptible |
| 1:10 | 1:200 | Susceptible |
| 1:10 | 1:500 | Susceptible |
| 1:10 | 1:1000 | Susceptible |

### Witch Hazel Extract

Witch hazel (Hamamelis) is a genus of flowering plants in the family Hamamelidaceae. Four species are found in North America (H. ovalis, H. virginiana and H. vernalis), and one species each is found in Japan (H. japonica) and China (H. mollis). The leaves and bark of the North American witch hazel, Hamamelis virginiana, may be used to produce an astringent decoction. This witch hazel decoction can be an extract in liquid form. This concentrated extract liquid has been FDA approved, for example, for topical application to the nose, anus and skin.

Witch hazel liquid extract/concentrate can include such components as calcium oxalate, gallotannins, and safrole, and chemicals found in the essential oil (carvacrol, eugenol). Witch hazel for use as a topical can be a liquid, semisolid ointment, cream, gel or salve, as examples. Witch hazel extracts and concentrates are widely sold by pharmacies, grocery stores, and others, and they serve many uses to ease discomfort and provide soothing sensations.

A particular witch hazel extract (WH) in the embodiments, determined to provide beneficial attributes, is marketed over the counter as a product named whISOBAX™ available from Staph-Off Biotech, Inc. at staphoff.com, as example. The whISOBAX™ product comprises about 12.66 mg of gallic acid equivalent/ml. The planktonic minimum bactericidal concentration/minimum inhibitory concentration (MBC/MIC) for whISOBAX™ is about 0.31/0.15 mg/ml gallic acid equivalence method (GAE) and the minimum inhibitory concentration (MIC) for biofilm trapped bacteria is about 0.47 mg/ml GAE [˜3× higher than the planktonic level].

Determined to be a particular anti-microbial component of the witch hazel extracts is the hamamelitannin molecule. The hamamelitannin molecule is substantially as follows:

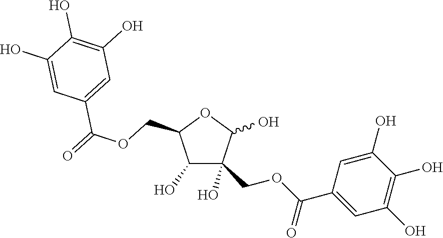 [SMILES: O=C(OC[C@H]1OC(O)[C@@](O)(COC(=O)c2cc(O)c(O)c(O)c2)[C@@H]1O)c1cc(O)c(O)c(O)c1]

Various analogues of hamamelitannin are possible and all are contemplated as suitable for the combinations in embodiments. Hamamelitannin acts as a quorum signaling inhibitor (QSI) that suppresses microbial biofilm formation and toxin production of bacteria.

Other anti-microbial active components of witch hazel include gallic acid and other phenolic compounds. Gallic acid, for example, may prevent bacterial growth by binding/disrupting cell membranes. The various components of witch hazel extract are effective in multiple gram negative and positive bacteria that commonly colonize indwelling urinary catheters and the like. The primary function of witch hazel extract where deposited and in antimicrobial actions is to block microbial attachment to devices or biological surfaces, block biofilm formation, erode existing biofilm and block microbial toxins from being produced, and lower the MIC of other anti-microbial agents.

### Acetohydroxamic Acid

Acetohydroxamic acid (AHA) is a urease enzyme (protein) inhibitor. Proteus and certain other urinary microbes make urease which alkalinize urine by splitting abundant urinary urea. Alkalinity decreases microbiocidal functionality of iodine and hydrogen peroxide as antimicrobials. Addition of AHA to combinations of aqueous iodine and/or aqueous hydrogen peroxide are found to block urease induced alkalinity and thereby improve the microbiocidal efficacy of Iodine and/or hydrogen peroxide as antimicrobials, particularly in presence of Proteus, Klebsiella and Pseudomonas and other microbial species that make and secrete urease.

### Combinations

In certain embodiments, combinations of AHA with acidic aqueous iodine and hydrogen peroxide are found to be particularly effective antimicrobial compositions. Aspirin (i.e., acetylsalicylic acid), acetic acid, benzoic acid, hydrochloric acid, or other acidic and tolerable agents, added to the combination, either to iodine or hydrogen peroxide, alone or together, decreases the pH of the solution. The lowered pH increases the concentration and microbiocidal effectiveness of molecular iodine [and decreases the ionic concentration of iodine]. The acidic aqueous solution is more efficacious than individual components in limiting microbial growth. Testing of the combination of aqueous ingredients reveals that no reactants, such as precipitates, flocculates, or color change (other than dilution), results.

In certain further embodiments, increasing the concentration and acidity of either or both iodine and hydrogen peroxide, while simultaneously minimizing microbe protective biofilm formation with WH, furthers antimicrobial efficacy of the iodine and hydrogen peroxide. In the combination of aqueous iodine and hydrogen peroxide with WH, the WH prevents and erodes microbe induced biofilm on cellular and prosthetic surfaces. Exceptional bench-top testing results have been obtained in antimicrobial action from the iodine, hydrogen peroxide and WH combination in the testing of multiple Proteus species. It is noteworthy that all Proteus strains [e.g., mirabilis, vulgaris, rettgeri, etc.] rapidly alkalinize bladder urine [via urease-induced splitting of dissolved urinary urea into alkaline by-products thereby inducing particulate calcium phosphate crystals. This functionality is prevented by acetohydroxamic acid dissolved in bladder urine.

In certain other embodiments, addition of AHA to the combination of either iodine, hydrogen peroxide, and/or WH, or combinations thereof, is effective to block microbe induced alkalinization of urine thereby enhancing potency of iodine and hydrogen peroxide as antimicrobials. Microbe induced alkalization (e.g., of urine) is unique to some microbial species that make and release the protein urease into urine when bacteria are present in the urine. Urine is sensitive to alkalinity because microbial urease reacts with dense concentrations of urea dissolved in urine. Urea in high blood concentrations is a toxin that can cause illness and even death. The addition of AHA into urine to limit the microbe induced alkalization is therefore beneficial and in combination with other agents yields unexpectedly favorable results in the combination.

### Silver Ions

Silver ions, as well as other metals, for nonexclusive example, copper, tin, zinc, lead and others, can also have biocidal effects. Further, various halogens, such as, for nonexclusive example, iodine, bromine, chlorine and others, may contribute to antibacterial action. The mechanism of action of metals and halogens is believed to involve penetration into bacterial cells, turning deoxyribonucleic acid (DNA) molecules into a condensed form and thereby hindering cell replication.

Silver colloid, for example, has been approved by FDA for topical and oral use. Silver colloids can be a preferred drinking water sterilant when used in 20-100 mcg/liter concentrations. Russian space craft employ silver colloids to recycle urine and other body fluids. Other metals and colloids of metals are therefore effective for sterilization as well as other antimicrobial effects.

The combination of witch hazel extract, with silver or other metal ion or colloid or halogen, has been determined in bench top testing to surprisingly serve as a more powerful antimicrobial than either agent alone. Testing in multiple dilutions, ranging from about 1:5, 1:10, 1:20, 1:40, 1:80 and others, indicates the efficacy of the combination against both gram+ and gram− microbial species.

A particular witch hazel extract, whISOBAX™, has about the following characteristics:

| Test | Specifications | | Method | Results |
|---|---|---|---|---|
| Identity | Hamamelis | | Macroscopy, | Pass |
| | virginiana L. | | microscopy and | |
| | bark extract | | chromatography | |
| Ethanol/Water | 50% 190 proof | | Measurement | Pass |
| ratio | EthOH/50% H2O | | by volume | |
| Density @ 20° | 0.93-0.96 | | Measurement by | 0.948 |
| | | | weight/volume | |
| Odor | Characteristic | | Examine | Pass |
| | witch hazel, mildly | | organoleptic | |
| | aromatic, free | | | |
| | from Acetous Odor | | | |
| Color | Red/brown | | Examine (visual | Pass |
| | | | inspection) | |
| Clarity | Transparent | | Examine (visual | Pass |
| | | | inspection) | |
| pH @ 25° C. | 4.0-6.0 | | Measurement | 5.35 |
| Specific gravity | 0.902-0.99 | | Measurement | 0.949 |
| @ 20° C. | | | | |
| Tannins | >10 | mg/ml | HPLC | 17.3 mg/ml |
| Total Yeast | <1000 | CFU/ml | USP <2021>, | Pass |
| and Mold | | | USP <2022> | |
| | | | Modified | |
| Aerobic | <10,000 | CFU/ml | USP <2021>, | Pass |
| Plate Count | | | USP <2022> | |
| | | | Modified | |
| Total | <10 | cfu/ml | USP <2021>, | Pass |
| Coliforms | | | USP <2022> | |
| | | | Modified | |

| | | | |
|---|---|---|---|
| Salmonella/ | Negative | USP <2021>, | Pass |
| E.coli/ | | USP <2022> | |
| S. aureus | | Modified | |
| Contaminants | <1.667/0.684/ | ICP-MS | <0.002/ |
| (Arsenic, | 0.0834/0.33 | (EPA 200.8 | 0.001/0.002/ |
| Cadmium, | 4 μg/ml (3 ml | [modified] | 0.005 μg/ml |
| Lead, Mercury) | daily dose) | | |

This witch hazel extract, or similar variants of it, were employed in combinations in certain non-exclusive embodiments.

In tests, whISOBAX™ was combined with silver ion colloid. A particular silver ion colloid in tests, determined to provide beneficial attributes, is marketed over the counter as a product named Ultra Silver™ available from UltraSilver.com or Amazon.com, as example. Both whISOBAX™ and Ultra Silver™ are FDA certified as dietary supplements.

A particular silver colloid, UltraSilver™, has about the following characteristics:

| | |
|---|---|
| Pharmaceutical grade silver | 10,000 PPM (1000 μg/l) |
| Steam distilled water | </=~1 \| |
| Protein | Balance |

This silver colloid, or similar variants of it or of other metal or of halogen colloids, were employed in combinations in certain non-exclusive embodiments.

Although nota necessity to the effectiveness of the combinations as antimicrobials, silver and other metals and halogens can be included to further enhance the combination of agents effectiveness as an antimicrobial.

### Examples

Iodine—Hydrogen Peroxide Combination:

Iodine and hydrogen peroxide combination kills microbes at greater dilutions (i.e., lower concentrations) that does either agent alone. Neither of these agents has previously been studied as a bladder additive or device coating, or similar internal or device application. Potency of both iodine and hydrogen peroxide are increased in an acidic liquid formulation and are reduced in an alkaline liquid formulation. Studies reveal no interactivity or reactivity of the two agent combination, at room or body temperatures.

Addition of Witch Hazel Extract

WH concentrate, and its components hamamelitannin and gallic acid, have been observed to effectively block and erode cellular surface and inanimate surface adherent microbes. The combination of WH (or components of it) with iodine and hydrogen peroxide prevent and strip away microbe enshrouded biofilm thereby exposing adherent microbes to the efficacious and enhanced antimicrobial effects (e.g., microbe killing effects) of iodine and hydrogen peroxide. The combination has been observed to exceed antimicrobial effects experienced with the individual components.

Addition of Acetohydroxamic Acid

In the foregoing varied combinations, acetohydroxamic acid (AHA) included in the combinations further blocks microbe induced alkalinization of urine. This increases the effectiveness of iodine and hydrogen peroxide, alone or in combination in a urinary application.

Other Additives

Silver, and other metals, and halogens may be additional or alternately included in the various combinations. These additives may have enhanced antimicrobial effectiveness in the presence of a quorum-signaling inhibitor [e.g., witch hazel components] or the like.

The foregoing components and combinations are employable as surface coatings, for example, on the skin, or on the surface of permanently implanted devices, and/or as and as liquid aqueous additives to surface of temporary replaceable devices like urinary catheters, and as a cleansing or rinse agent on biological or inanimate surfaces or the like, thereof. In particular, the combinations with WH are particularly effective as antimicrobials on surfaces of urinary catheters and other devices, as well as skin and wounds. The combinations with AHA further enhance the effectiveness of other components by restricting microbe induced alkalinization, such as in urine. Additional, but not necessarily requisite, combination of the foregoing with other metals and/or halogens, such as in silver colloid, appear to be even more efficacious in combating bacterial infection. In any event, the various combinations appear to be more effective as antimicrobials than would be expected from results of each component acting alone.

Patients with indwelling catheters, for non-exclusive example, may receive at least one or twice daily [AM/PM] instillations of a combination of one or more of the various combinations as a rinse into the urinary bladder for at least two days and possibly for the duration of use of the indwelling catheter (or other device, as applicable). Following each instillation, bladder drainage will be capped/clamped to tolerance. Unclamping and drainage of bladder urine into collection bags will be initiated by the patient or caregiver when symptoms dictate a need for urine drainage. The above agent delivery methodology mimics the twice daily dental cleansing methodology that has improved dental hygiene and preserved dental functionality regardless of age.

A urine specimen will be collected each morning from the patient's indwelling catheter. The indwelling and/or the continuous draining catheter may be clamped for 10-15 minutes prior to urine collection if needed. After the urine specimen is collected for urine culture testing, the indwelling catheter will be irrigated with 20+ml of sterile water or saline to remove any mucous, blood, blood clots or crystalline debris from the bladder lumen. This irrigant will be discarded. Promptly thereafter, an applicable combination additive will be instilled into the bladder lumen and the catheter will be clamped to tolerance to allow the additive to remain in the bladder lumen for multiple hours. This combination additive instilled into the bladder is to remain within the bladder until the patient has urge to urinate. The patient or caregiver will then unclamp/uncap drainage tube and allow the bladder to drain into a commode or collection bag. There is no need for additive placement into the bladder lumen except for the early morning and bed-time time periods at this time. Increase or decrease in the number and timing of daily bladder instillations may be variable according to implementation. Each of the collected urine specimens will have a label for patient ID #, date, time of specimen collection and earlier time that additive was added to the bladder lumen. Additional urine specimen(s) may or may not be collected for infection testing if symptoms prompt such concerns. The specimens will be refrigerated in a research refrigerator at the local institution and then transported to a certified lab for microbiology testing.

As further protocol detail, an initial screening visit will be held. Informed consent and demographic date will be obtained. All antibiotics will be stopped at least two days prior to urine collection for the pre-treatment urine culture and for duration of the protocol procedure.

A second screening visit will then be held at least two days after the initial screening visit. A urine culture will be performed at this second screening visit.

Thereafter, in days 1, 2, and 3, after beginning urine culture from the second screening,
a. A urine dipstick test will be performed every morning.
b. The urine collection bag will be emptied prior to each bladder instillation.
c. The applicable combination shall be injected into and through the indwelling Foley catheter into the bladder in the morning and in the evening. Following instillation, the catheter will be capped, clamped or sealed in order to keep the additive within the bladder until the bladder is uncomfortably full.
e. All patients will be encouraged to keep the catheter clamped to tolerance after instillation of above fluids.
f. 10+ cc of urine will be collected directly from the indwelling bladder catheter each morning per day immediately before installation of the morning or evening combination agent. Early in day 1 collection will be made of urine specimen collection #1, followed by installation #1, and a second urine specimen collection #2 followed by installation #2 shall be made in the early AM of day 2. Third and fourth installations of the combination shall similarly be made on days 3 and 4 (mornings) Daily side effects will be logged and discussed with the patient. Single early AM bladder instillations are anticipated to eradicate the original microbial growth in the bladder urine. If single early AM instillations of the bladder additive do not sterilize bladder urine then twice daily instillations of the additive [i.e. early sunrise AM and late sunset PM] shall be offered.

In non-exclusive alternatives, the combination of iodine and/or hydrogen peroxide, particularly each in aqueous form and acidified by addition of a suitable acid (such as aspirin, hydrochloric acid, acetic acid, or benzoic acid or other), may be combined with AHA and/or with witch hazel concentrate or its agents, i.e., hamamelitannin and/or gallic acid compositions, as liquid or gel forms for rinse and/or topical use as effective antimicrobial agents. Additionally, metal or halogen colloid, such as silver colloid, or witch hazel can be included in any of the combinations to enhance antimicrobial quorum inhibition and antimicrobial effectiveness.

Non-exclusive embodiments may take form of liquid, gel, or device coating or the like, for application to surfaces of devices or body organs or spaces or cavities for rinse or cleansing of the body or device. Additionally, the individual agents and/or combinations can be impregnated or disposed in or on tape, cloth, device, or other surfaces. The combination can be placed as a coating on devices, such as a catheter or other medical device made of polyethylene, silicone, composites, or other materials. Variations of viscosity and flow characteristics, as well as elution and retention properties, are possible in the embodiments. In certain non-exclusive alternatives, combinations of the embodiments are introduced by instillation into bodily cavities, such as bladder lumen, or other vessels or other body parts, or as coating or deposited ‘additive’ on body part, coating or deposition, as on a medical device, such as a urinary catheter or other device, or as topical application to the skin or bodily area, cavity or organ.

Of course, a wide variety of other variations are possible.

In the foregoing, therefore, the invention has been described with reference to specific embodiments. One of ordinary skill in the art will appreciate, however, that various modifications, substitutions, deletions, and additions can be made without departing from the scope of the invention. Accordingly, the specification and figures are to be regarded in an illustrative rather than a restrictive sense, and all such modifications substitutions, deletions, and additions are intended to be included within the scope of the invention. Any benefits, advantages, or solutions to problems that may have been described above with regard to specific embodiments, as well as device(s), connection(s), step(s) and element(s) that may cause any benefit, advantage, or solution to occur or become more pronounced, are not to be construed as a critical, required, or essential feature or element.

What is claimed is:

1. A biological or inanimate surface cleansing, rinsing and antimicrobial composition, comprising:
iodine; and
hydrogen peroxide.

2. The composition of claim 1, wherein the hydrogen peroxide is aqueous.

3. The composition of claim 2, wherein the iodine is selected from the group consisting of: aqueous or ethanolic [e.g., tincture of iodine] and mixture of ionic or elemental iodine [e.g., known as Lugol's solution].

4. The composition of claim 3, further comprising:
an acidic agent for lowering pH of the aqueous and/or ethanolic and/or iodinated solution.

5. The composition of claim 1, further comprising:
witch hazel extract.

6. The composition of claim 4, further comprising:
witch hazel extract.

7. The composition of claim 5, wherein the witch hazel extract comprises hamamelitannin.

8. The composition of claim 5, wherein the witch hazel extract comprises gallic acid.

9. The composition of claim 6, wherein the witch hazel extract comprises hamamelitannin.

10. The composition of claim 6, wherein the witch hazel extract comprises gallic acid.

11. The composition of claim 4, wherein the acidic agent is aspirin.

12. The composition of claim 5, wherein the acidic agent is aspirin.

13. The composition of claim 6, wherein the acidic agent is aspirin.

14. The composition of claim 12, further comprising an agent selected from the group consisting of: silver ion, metal, halogen and combinations of these.

15. The composition of claim 13, further comprising an agent selected from the group consisting of: silver ion, metal, halogen and combinations of these.

16. A method of antimicrobial action, comprising:
providing an aqueous iodine;
providing an aqueous hydrogen peroxide to the aqueous iodine for a first composition;
acidifying the first composition.

17. The method of claim 16, wherein the acidifying includes addition of an acid to the first combination, selected from the group consisting of: aspirin, acetohydroxamic acid, hydrochloric acid, other human tolerable acid, and combinations of these.

18. The method of claim 17, further comprising:
combining a witch hazel extract with the first combination to obtain a second combination.

19. The method of claim 18, wherein the witch hazel extract comprises hamamelitannin.

20. The method of claim 19, wherein the witch hazel extract comprises gallic acid.

21. The method of claim 16, comprising:
applying the first combination to a body organ or tissue.

22. The method of claim 16, comprising:
applying the first combination to a device for insertion into body tissue. or body organ.

23. The method of claim 18, comprising:
applying the second combination to a body tissue or body organ.

24. The method of claim 16, comprising:
applying the second combination to a device for insertion in body tissue or body organ.

25. The method of claim 16, wherein the aqueous iodine is acidic and combination with the aqueous hydrogen peroxide forms the acidifying the first composition.

26. The method of claim 16, wherein the aqueous hydrogen peroxide is acidic and combination with the aqueous iodine forms the acidifying the first composition.

27. The method of claim 25, wherein the aqueous hydrogen peroxide is acidic and combination with the aqueous iodine forms the acidifying the first composition.
